(19) United States
(12) Patent Application Publication
(10) Pub. No.: US 2011/0077614 A1
(43) Pub. Date: Mar. 31, 2011

(54) DEVICE AND METHOD FOR PATIENT ACTIVATED BOLUS ADMINISTRATION

(76) Inventor: Ofer Shay, Kfar Vradim (IL)

(21) Appl. No.: 12/446,241

(22) PCT Filed: Oct. 21, 2007
(86) PCT No.: PCT/IL07/01260

§ 371 Date: Dec. 15, 2010

Related U.S. Application Data
(60) Provisional application No. 60/852,662, filed on Oct. 19, 2006.

(51) Int. Cl. A61M 5/168; A61M 5/142

(52) U.S. Cl. 604/500; 604/151; 604/182

(57) ABSTRACT

A device and method to accept a setting of a maximum volume of a drug that is introduced into a subcutaneous channel upon a patient's direction, while retaining a pre-designated flow of the drug to the channel over an extended period after accounting for such maximum volume.

### FIELD OF THE INVENTION

The device and method relate to a patient-activated administration of a quantity of therapeutic solution inserted to the body intravenously or subcutaneously. More specifically, the application relates to an improved device and method for the activation of a supplementary dose volume of medication to the dose of continuous flow controlled infusion therapy.

### BACKGROUND OF THE INVENTION

Certain procedures or treatments, such as post-operative care, pain management, oncology, anti-biotic and other therapies call for a catheter to remain inside a patient body over an extended period of time at a continuous, designated rate or intermittently. Some therapies permit a patient to increase a flow or dosage at a particular time through the release of a increased volume of medication into the infusion administration. However, the therapy may nonetheless require that the total volume of the drug released into a subcutaneous channel remain constant over a designated period, such that a release of a greater volume at one or more points during an interval, be compensated by a release of smaller volumes at other points during the interval. In addition, some therapies set a maximum volume of a material that may be released by a patient action in a given period. For example, in some cases, once a patient triggers a release of an increased volume of an analgesic, there may be imposed a ‘lock out’ period during which the patient may not trigger a further release of the analgesic, or during which no further analgesic may flow into the intravenous channel.

Frequently, a doctor or practitioner sets a rate of a continuous flow of a medicine, and such medicine is introduced or pumped from a container by a pressure flow or other pump. A doctor may also want set a maximum volume or bolus that a patient can trigger, and a lock out period that may follow the bolus activation. The doctor may want to secure such setting against tampering by a patient.

### BRIEF SUMMARY OF THE FIGURES

The subject matter regarded as the invention is particularly pointed out and distinctly claimed in the concluding portion of the specification. The invention, however, both as to organization and method of operation, together with features and advantages thereof, may best be understood by reference to the following detailed description when read with the accompanied drawings in which:

FIG. 1 is an exploded view of components of a device in accordance with an embodiment of the invention;

FIG. 2 is a diagram of a base component of a device in accordance with an embodiment of the invention;

FIGS. 3A, 3B, 3C, 3D and 3E are diagrams of segments of the activator of a device in accordance with an embodiment of the invention;

FIG. 4 is a diagram of a reservoir with an inlet and an outlet in accordance with an embodiment of the invention;

FIGS. 5A, 5B and 5C are diagrams of a cover of a device showing a locking pin holster in accordance with an embodiment of the invention;

FIG. 6A shows a locking pin, and FIGS. 6B and 6C show the locking pin in a holster in an open and close position respectively, in accordance with an embodiment of the invention;

FIG. 7A and 7B show a front of a device without a cover and with a button and without, respectively in accordance with an embodiment of the invention;

FIG. 8 is a diagram of a device in a see-through view, attached to a wrist band in accordance with an embodiment of the invention;

FIG. 9A shows a device before a bolus volume setting has been selected and a security pin activated, and FIG. 9B shows a device after a bolus volume setting has been set and a security pin activated in accordance with an embodiment of the invention; and

FIG. 10 shows a drug administration system in which a device may be included in accordance with an embodiment of the invention.

### DETAILED DESCRIPTION OF THE EMBODIMENTS

In the following description, various embodiments of the invention will be described. For purposes of explanation, specific examples are set forth in order to provide a thorough understanding of at least one embodiment of the invention. However, it will also be apparent to one skilled in the art that other embodiments of the invention are not limited to the examples described herein. Furthermore, well-known features may be omitted or simplified in order not to obscure embodiments of the invention described herein.

As used herein, the terms “therapeutic agent,” “therapeutic material,” “active material,” “drug”, “medicine” and similar terms include in addition to their regular meanings, any therapeutic agent or active material, such as drugs, genetic materials, and biological materials. Suitable genetic materials include, but are not limited to, DNA or RNA, such as, without limitation, DNA/RNA encoding a useful protein, DNA/RNA intended to be inserted into a human body including viral vectors and non-viral vectors, and RNAi (RNA interfering sequences). Suitable viral vectors include, for example, adenoviruses, gutted adenoviruses, adeno-associated viruses, retroviruses, alpha viruses (Semliki Forest, Sindbis, etc.), lentiviruses, herpes simplex viruses, ex vivo modified and unmodified cells (e.g., stem cells, fibroblasts, myoblasts, satellite cells, pericytes, cardiomyocytes, skeletal myocytes, macrophage), replication competent viruses (e.g., ONYX-015), and hybrid vectors. Suitable non-viral vectors include, for example, artificial chromosomes and mini-chromosomes, plasmid DNA vectors (e.g., pCOR), cationic polymers (e.g., polyethyleneimine, polyethyleneimine (PEI)) graft copolymers (e.g., polyether-PEI and polyethylene oxide-PEI), neutral polymers PVP, SP1017 (SUPRATEK), lipids or lipoplexes, nanoparticles and microparticles with and without targeting sequences such as the protein transduction domain (PTD). Suitable biological materials include, but are not limited to, cells, yeasts, bacteria, proteins, peptides, cytokines, and hormones. Examples of suitable peptides and proteins include growth factors (e.g., FGF, FGF-1, FGF-2, VEGF, Endothelial Mitogenic Growth Factors, and epidermal growth factors, transforming growth factor .alpha. and .beta., platelet derived endothelial growth factor, platelet derived growth factor, tumor necrosis factor .alpha., hepatocyte growth factor and insulin-like growth factor), transcription factors, proteinkinases, CDK inhibitors, thymidine kinase, and bone morphogenic proteins (BMP's), such as BMP-2, BMP-3, BMP-4, BMP-5, BMP-6 (Vgr-1), BMP-7 (OP-1), BMP-8. BMP-9, BMP-10, BMP-11, BMP-12, BMP-13, BMP-14, BMP-15, and BMP-16. Currently preferred BMP's are BMP-2, BMP-3, BMP-4, BMP-5, BMP-6, and BMP-7. These dimeric proteins can be provided as homodimers, heterodimers, or combinations thereof, alone or together with other molecules. Cells can be of human origin (autologous or allogeneic) or from an animal source (xenogeneic), genetically engineered, if desired, to deliver proteins of interest at a desired site. The delivery media can be formulated as needed to maintain cell function and viability. Cells include, for example, whole bone marrow, bone marrow derived mono-nuclear cells, progenitor cells (e.g., endothelial progentitor cells), stem cells (e.g., mesenchymal, hematopoietic, neuronal), pluripotent stem cells, fibroblasts, macrophage, and satellite cells. The term “therapeutic agent” and similar terms also includes non-genetic agents, such as: analgesics, anti-thrombogenic agents such as heparin, heparin derivatives, urokinase, and PPack (dextrophenylalanine proline arginine chloromethylketone); anti-proliferative agents such as enoxaprin, angiopeptin, or monoclonal antibodies capable of blocking smooth muscle cell proliferation, hirudin, and acetylsalicylic acid, amlodipine and doxazosin; anti-inflammatory agents such as glucocorticoids, betamethasone, dexamethasone, prednisolone, corticosterone, budesonide, estrogen, sulfasalazine, and mesalamine; antineoplastic/antiproliferative/anti-miotic agents such as paclitaxel, 5-fluorouracil, cisplatin, vinblastine, vincristine, epothilones, methotrexate, azathioprine, adriamycin and mutamycin; endostatin, angiostatin and thymidine kinase inhibitors, taxol and its analogs or derivatives; anesthetic agents such as lidocaine, bupivacaine, and ropivacaine; anti-coagulants such as D-Phe-Pro-Arg chloromethyl keton, an RGD peptide-containing compound, heparin, antithrombin compounds, platelet receptor antagonists, anti-thrombin antibodies, anti-platelet receptor antibodies, aspirin, dipyridamole, protamine, hirudin, prostaglandin inhibitors, platelet inhibitors and tick antiplatelet peptides; vascular cell growth promotors such as growth factors, Vascular Endothelial Growth Factors (VEGF, all types including VEGF-2), growth factor receptors, transcriptional activators, Insulin Growth Factor (IGF), Hepatocyte Growth Factor (HGF), and translational promotors; vascular cell growth inhibitors such as antiproliferative agents, growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; cholesterol-lowering agents, vasodilating agents, and agents which interfere with endogenous vasoactive mechanisms; anti-oxidants, such as probucol; antibiotic agents, such as penicillin, cefoxitin, oxacillin, tobranycin; angiogenic substances, such as acidic and basic fibrobrast growth factors, estrogen including estradiol (E2), estriol (E3) and 17-Beta Estradiol; and drugs for heart failure, such as digoxin, beta-blockers, angiotensin-converting enzyme (ACE) inhibitors including captopril and enalopril.

In some embodiments, a medication or drug as used in this paper may include a medication that is suitable to be administered through infusion.

An intravenous or subcutaneous channel may, in addition to its regular meaning include a channel such as a tube for delivery of a mass, volume or bolus of therapeutic material into an intravenous, intra-dermal, intra-muscular or other channel into which medicines may be introduced or channeled into a body.

A bolus or volume may, in addition to its regular meaning, include a fluidic, semi-fluidic, suspended solid or solid of one or more therapeutic materials.

In some embodiments, a lock out period may be determined by a formula such as V/FR, where FR is a flow rate at the inlet tube of a device, and V is an effective volume of the reservoir of the device. Other ways to determine a lock out period are possible.

Reference is made to FIG. 10, a drug administration system in which a patient activated device may be included in accordance with an embodiment of the invention. In some embodiments, device 1000 may be part of or used with a drug administration system. The system may include a container or source of the drug being introduced into the system and a pump or other flow regulator by which a doctor or practitioner may set a flow of a drug from the container into a channel so that a set amount of the drug is introduced into the channel over a designated interval. The system may include one or more filters, device 1000 in accordance with an embodiment of the invention, one or more check valves and an exit port through which the drug enters a body area of a patient. In some embodiments, and absent patient activation of a pump or trigger in or connected with device 1000, a drug may flow through the system and through device 1000 at a rate designated by a doctor or practitioner in the pump or flow regulator. In some embodiments, the activation by a patient of device 1000 may not increase the total amount of the drug introduced or delivered from the container into the system, but rather the amount or volume of the drug delivered at a particular moment and upon the activation of trigger or pump in or connected to device 1000. In some embodiments, a drug may be supplied to device 1000 by a fixed flow rate restrictor or by a multi-flow rate flow regulator.

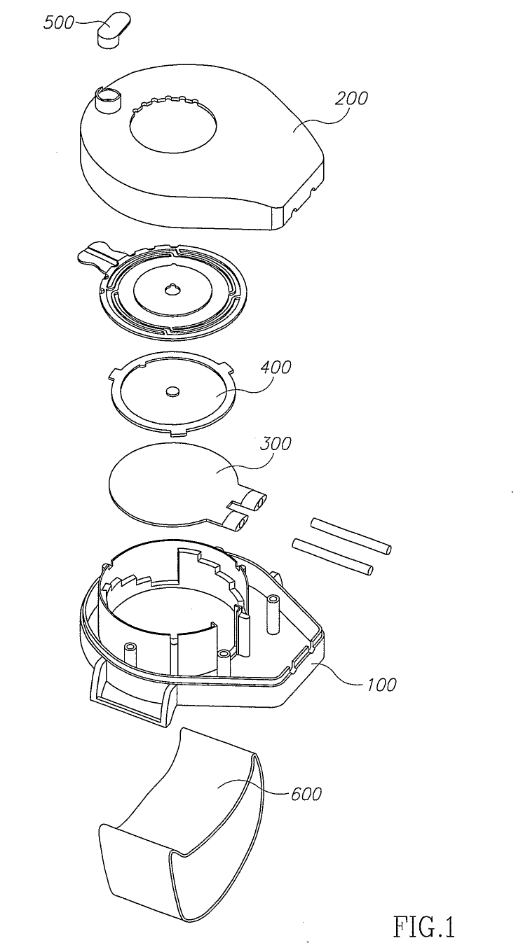

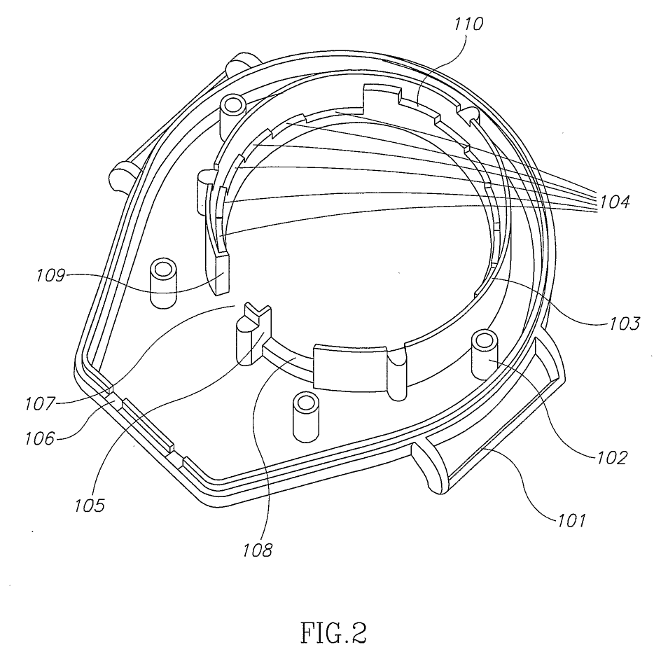

Reference is made to FIG. 1, an exploded view of components of a device in accordance with an embodiment of the invention, and to FIG. 2, a diagram of a base component of a device in accordance with an embodiment of the invention. Base 100 may be constructed of for example rigid plastic or other suitable material. A circular cylinder 103 may be positioned perpendicular to the bottom of base 100. The inner hollow created by cylinder 103 may hold reservoir 300. Openings 107 and 108 on cylinder 103 may accommodate reservoir inlet and exit tubes while pillar 105 serves as a clockwise stopper to the activator 400 and to prevent reservoir 300 from sliding out of cylinder 103. Pillar 109 may serve as a counterclockwise stopper to activator 400. Terraced cylinder 104 may be positioned concentrically to cylinder 103 at the inner circular edge of cylinder 103. Cylinder 104 may be divided into several repeating and essentially similar segments, such as for example three segments, and such segments may include several steps 110 having width and height that are the same as the corresponding steps 110 in the other segments. Steps 110 on inner cylinder 104 may function as bottom stoppers to activator 400 such that upon activation, activator 400 may be depressed no further than the step 110 to which device 1000 is set. The number of steps 110 in the segments may be equal to the number of volume settings which device 1000 may expel in a single activation. Some embodiments may include six settings though other number of settings and volumes are possible.

In some embodiments, wrist belt 600 may be threaded through eyelets 101 to provide a patient with easy access to device 1000, and so that device 1000 is portable with the patient.

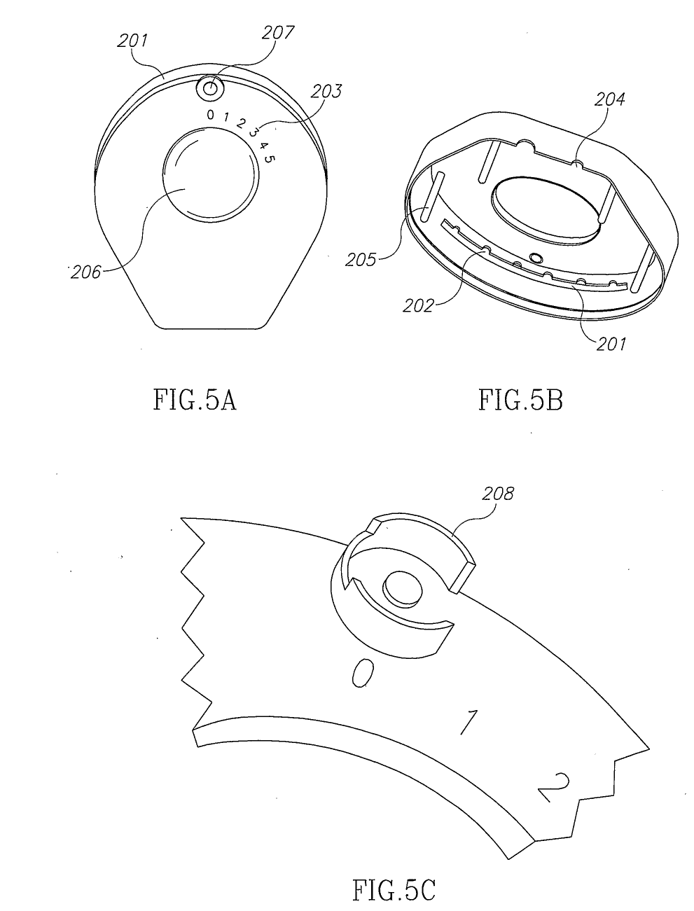

Dents 106 may securely hold the inlet and exit tubes. Dents 106 may be matched with corresponding and opposite dents 204 (as appear in FIG. 5B) in cover 200 to surround inlet and outlet tubes from above and below. Hollow posts 102 may connect base 100 to cover 200 through compatible pins 205 as appear in FIG. 5B.

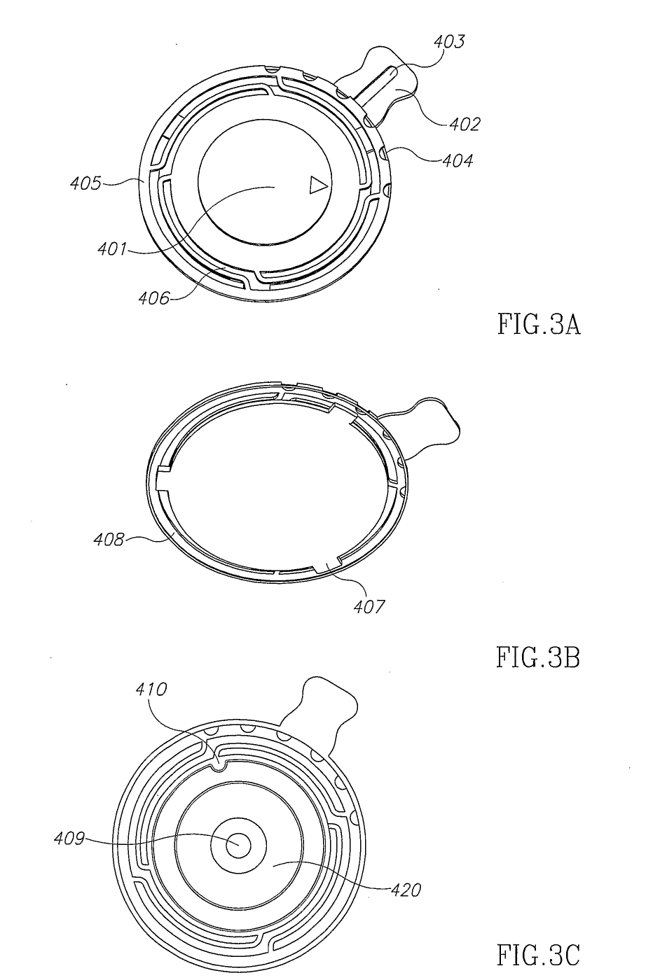

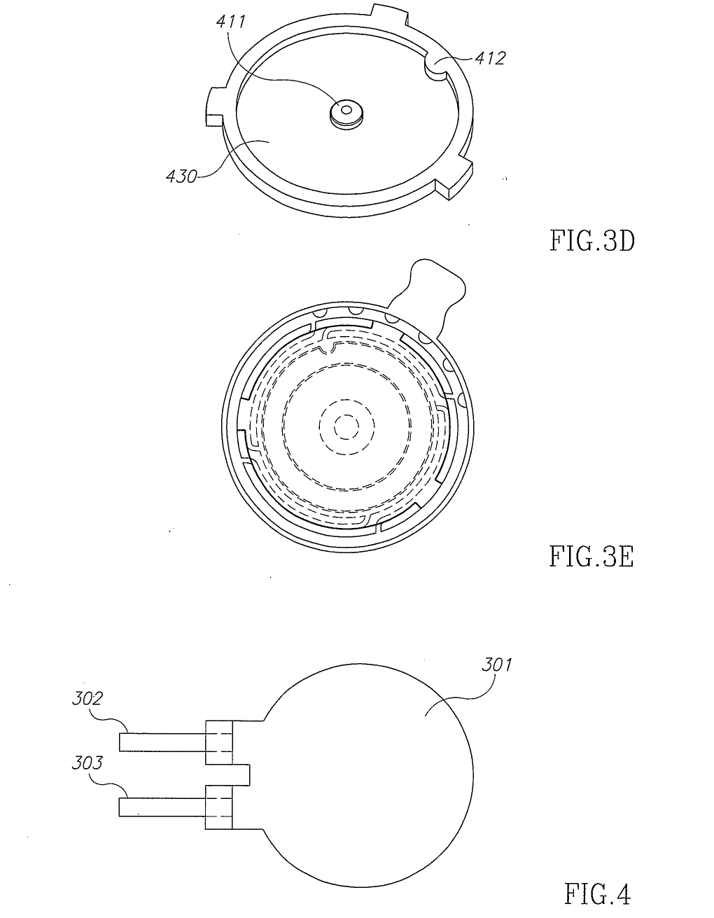

Reference is made to FIGS. 3A, 3B, 3C, 3D and 3E, diagrams of segments of the activator of a device in accordance with an embodiment of the invention. Activator 400 may be constructed of one or more parts. Button 420 of activator 400 may include a return spring as an integrated part of button 420 or may include a spring, such as a coil or other spring, as a separate component. Button 420 may include segments or components such as button 401 that may include a marking to indicate a setting of device 1000, a spring that made include two or more leaves 406, external ring 405 and arm 402 to dial a volume setting for a dosage expelled by device 1000.

In some embodiments, the activator may include two parts, such as a button 420 and stopper 430. Such parts may be constructed of plastic or other suitable materials. Button 420 may include projection 403 at the upper face of arm 402 to secure and hold the selected volume setting. Dents 404 at the external circumference of ring 405 may facilitate a locking of a volume setting of device 1000 to prevent tampering or alteration of such setting by a patient or others, once the setting has been selected. Circular projection 408 at the lower external diameter of ring 405 may center the activator 400 over cylinder 103 of base 100, allowing circular movement between the different volume settings. Three projections 407 of stopper 430 may prevent vertical movement of activator 400 and its corresponding compression of reservoir container 301, when set over the suitable step 110 of cylinder 104. Dents 409 and 410 on the bottom of button 420 and projections 411 and 412 on the top of stopper 430 may orient parts when assembled.

Reference is made to FIG. 4, a diagram of a reservoir with an inlet and an outlet in accordance with an embodiment of the invention. Reservoir 300 may include three parts, namely reservoir container 301, inlet 302 and outlet 303, though fewer or greater number of parts are possible. One or more of inlet 302 and outlet 303 may be equipped with one-way valves to avoid backflow. The reservoir can be “flexible” or “semi-rigid”. Flexible reservoir container 301 may be constructed of thin plastic sheets such as PVC, PU or PE. Other materials may be used. In some embodiments, reservoir container 301 may be semi-rigid and fashioned as a molded balloon of a suitable material.

Reference is made to FIGS. 5A, 5B and 5C, diagrams of a cover of a device showing a locking pin holster in accordance with an embodiment of the invention. Cover 200 may be constructed of rigid plastic or other suitable materials.

Central hole 206 may locate the central section of button 401 of activator 400. Slot 201 may enable circular movement of arm 402 to select a volume setting. Dents 202 together with projection 403 may ease the positioning of activator 400 at the correct location when dialed to select the volume, and may secure it from further movement. Indicator 203 may indicate the possible volumetric or other settings for device 1000.

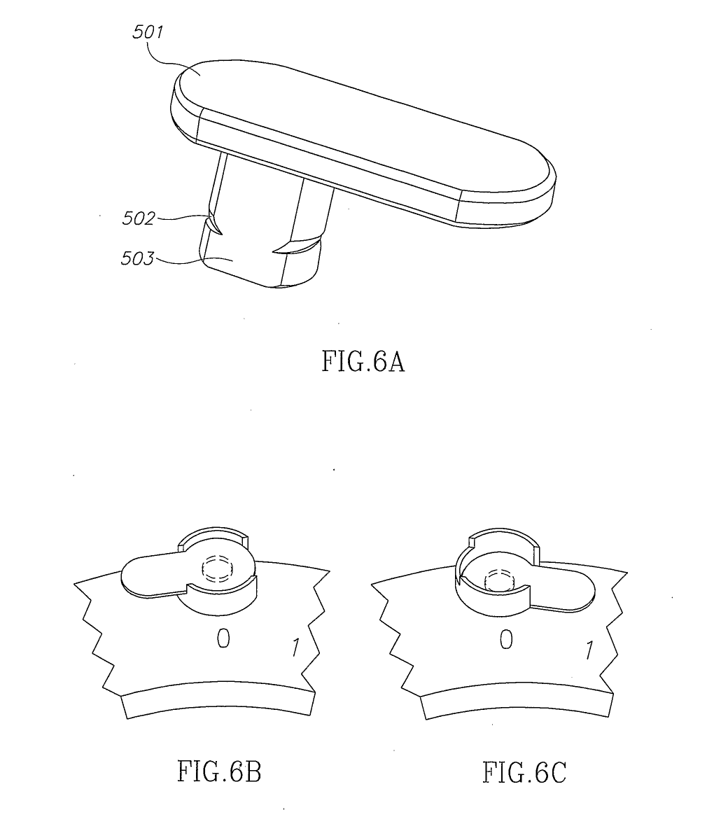

Reference is made to FIG. 6A, a locking pin, and FIGS. 6B and 6C, a locking pin in a holster in an open and close position respectively, in accordance with an embodiment of the invention. Cylinder 208 and hole 207 may hold, protect and guide locking pin 500. Locking pin 500 may be constructed of rigid plastic or other suitable material. In an un-locked position, pin 500 may be positioned on cover 200 so arm 501 is positioned over cylinder 208 while rod 503 may be positioned in hole 207.

To permanently lock the selected volume, arm 501 may be turned 180° clockwise and then pushed down to a locked position. In such position the lower edge of rod 503 is positioned in one of dents 404, dent 502 may be clicked into hole 207 to lock the setting. Other methods of locking a selected volume can be made so that no alteration of the selected volume are possible. In some embodiments, such permanent locking may require that device 1000 be disposable and suitable for only one use.

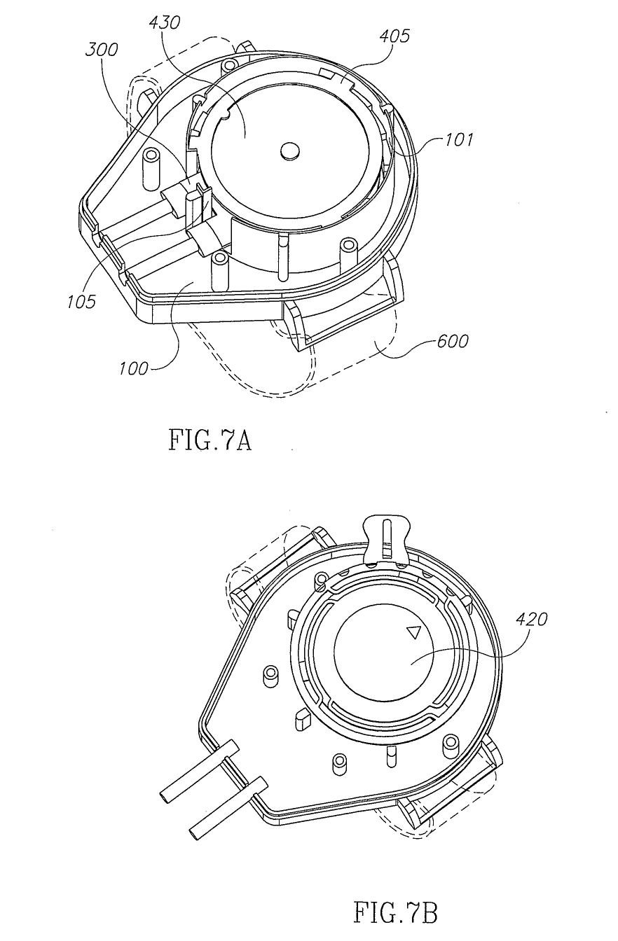

Reference is made to FIG. 7A and 7B, a front a device with a without a cover in accordance with an embodiment of the invention.

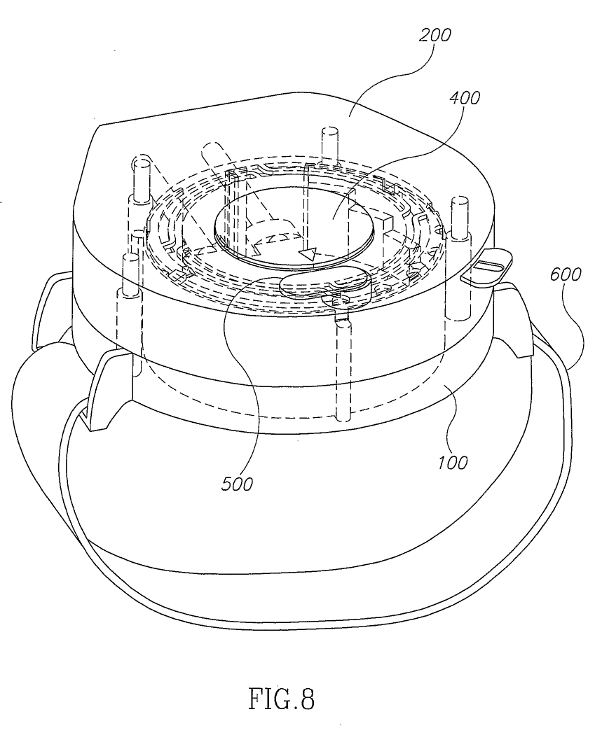

Reference is made to FIG. 8, a diagram of a device in a see-through view, attached to a wrist band in accordance with an embodiment of the invention.

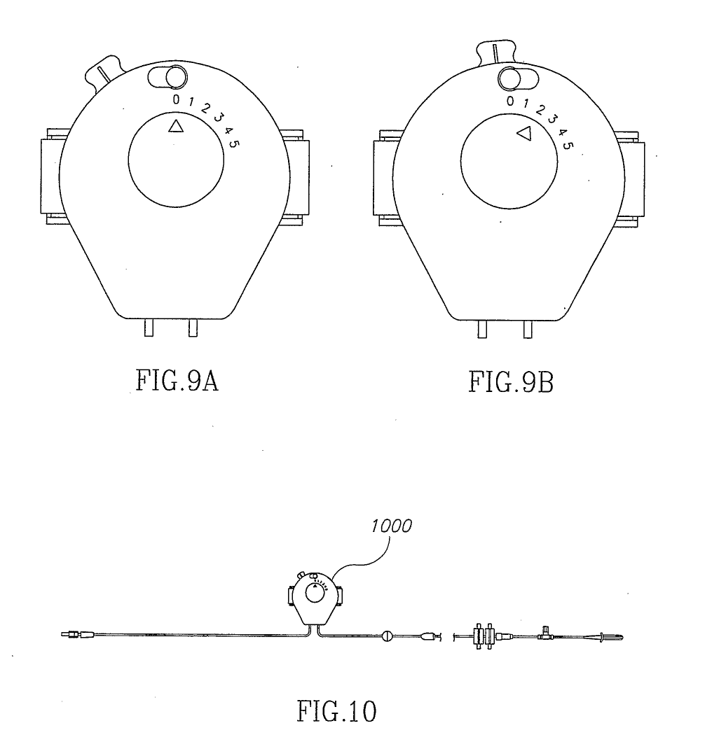

Reference is made to FIG. 9A, a device before a volume setting has been selected and a security pin activated, and to FIG. 9B, a device after a volume setting has been set and a security pin activated in accordance with an embodiment of the invention.

In some embodiments, volume settings may range from 0 ml. to 5 ml. with various steps such as 0.5 ml. or 1.5 ml. etc. Other settings, number of settings and volumes are possible.

In some embodiments, the device may be held or fastened other than on a user's wrist.

In operation, device 1000 may be linked to a container of a drug to be administered. A pressure pump or other device or force may release the drug into a line or channel to which device 1000 is connected. In some embodiments, reservoir 300 in device 1000 may be primed to be for example full of the drug being administered at the time that the connection to the patient is initiated or at some other time. A user such as a patient may activate or press a pump or release mechanism that may be included in or connected to device 1000 so that some or all of the contents of reservoir 300 are released by device 1000 further into the channel and into a body of the patient. The volume of medicine that may be released by the user's activation may be set in advance by a practitioner from among a choice of volumes. Once the user has activated the release of a drug from device 1000, the reservoir may be emptied or at least partially emptied. The user will therefore not be able to release additional amounts of the drug into his body until the reservoir 300 refills at the rate provided by the pressure pump that is releasing the drug from the container. When the reservoir 300 is full, the device 1000 passes the drug along at the same rate as its release into the system from the container.

Device 1000 may include a security lock to prevent a user from altering a volume setting that may be released from the device in a single activation, once such setting has been selected by for example a practitioner. In some embodiments, once a volume setting has been made and locked, no further changes to such setting may be made.

In some embodiments, an inlet that may be connected to a reservoir of device 1000 may accept a flow of a drug at a pre-defined rate from for example a container of the drug and a pressure system or pump that may deliver the drug from the container to the reservoir 300. In some embodiments, a separate pump or force-exertion device that may be connected to device 1000 may exert a force such as pressure upon.reservoir 300 of device 1000 to expel up to a maximum of a pre-designated volume of the drug from reservoir 300 to an outlet 303 channel of device 1000. In some embodiments, the maximum pre-designated volume to be expelled from reservoir 300 upon activation of the force on reservoir 300 may be set in advance by for example a practitioner. In some embodiments, a tamper-prevention mechanism may disable a means to accept a maximum volume setting once such setting has been accepted so that a maximum volume setting can only be made once.

In some embodiments, once a volume of a drug has been expelled from reservoir 300 and delivered to an outlet 303 of device 1000 and into a channel leading to a body, reservoir 300 may refill at the pre-defined rate from the container, and further expelling of volumes of the drug may be limited by such rate of refilling, such that the total output of the drug from device 1000 over an extended period is equal to the pre-defined rate delivered from the container to reservoir 300 by way of the inlet.

In some embodiments, when reservoir 300 is full, and a force such as pressure is not exerted on reservoir 300, the drug may flow from inlet 302 to outlet 303 at the predefined rate as is delivered from the container, so that a patient receives the drug at the pre-defined rate over the extended period after giving effect to, or after including the volume that may have been expelled when the force or pressure from for example the pump was activated.

In some embodiments, a device including reservoir 300, a pump or pressure exerting component, an inlet 302, an outlet 303, and setting accepting mechanism may be housed in a single unit that may be worn or otherwise attached to a patient on for example a wrist band or with a clip to the patients finger, clothing or other body part, and the force exerting mechanism may be activated by the patient who is to receive the drug.

In some embodiments, the maximum volume that may be expelled from reservoir may be set from among several possible volume settings.

In some embodiments, accepting a setting of a maximum volume that is to be expelled from reservoir 300 may include limiting the maximum reduction in the volume of reservoir 300 that is caused by the force exerted on it. The setting on the restriction of the volume may be variable from among a fixed number of possible volumes or a continuous number of possible volumes. For example, if a setting of 2.5 ml is accepted, and reservoir 300 holds a maximum of 5 ml., then the setting mechanism may restrict the amount that the volume of reservoir 300 is reduced when the force is exerted upon it. In some embodiments, this may mean that the pump can only be depressed partially, so that the reservoir is only emptied half way.

In some embodiments, a volume of a drug may be introduced by the device only when for example a patient activates the force such as pressure on the reservoir. In such embodiments, a pressure-triggered valve of for example 3 atmospheres or some other pressure setting may be connected to outlet 313, such that the outlet is opened only when a force such as pressure is activated on the device. When the force is not activated, the valve may be closed and the reservoir may fill until the next activation. In some embodiments, a mechanical force such as pressure that may be exerted manually by a patient on a button of the device may open the pressure valve and deliver a volume of drug.

It will be appreciated by persons skilled in the art that embodiments of the invention are not limited by what has been particularly shown and described hereinabove. Rather the scope of at least one embodiment of the invention is defined by the claims below.

I claim:

1. A device comprising:
a reservoir to accept a flow of a medicine at a pre-defined rate;
a pump to expel up to a pre-designated volume of said medicine from said reservoir;
a volume setting means to accept said pre-designated volume setting for said expelling from among a plurality of volume settings; and
an outlet to direct a flow of said medicine from said reservoir to a channel to a body at said pre-defined rate when said reservoir is full and at a volume of up to said pre-designated volume when said pump is activated.

2. The device as in claim 1, comprising a tamper prevention means to prevent an alteration of said pre-designated volume setting.

3. The device as in claim 2, wherein said tamper prevention means comprises a means to set said pre-designated volume only once.

4. The device as in claim 1, comprising an inlet to direct said flow to said reservoir at said pre-defined rate.

5. The device as in claim 1, wherein said pre-designated volume is between 0.5 ml and 5 ml.

6. The device as in claim 1, comprising a single housing suitable to contain said device and suitable for carrying on a body of a user.

7. The device as in claim 1, wherein said pump is activated by a recipient of said medicine through said channel.

8. A method comprising:
accepting a setting of a maximum volume of medicine to be expelled into an exit channel from a reservoir upon activation of a volume delivery means, said maximum volume from among plurality of volume settings;
selecting a flow rate of said medicine to flow through said exit channel over a pre-defined period, said flow rate to include said volume expelled by said pump during said period.

9. The method as in claim 8, wherein said accepting said setting comprises disabling a setting accepting mechanism.

10. The method as in claim 8, activating said volume delivery means by exerting pressure upon said reservoir.

11. The method as in claim 8, wherein said accepting comprises limiting a reduction in volume of said reservoir that is achieved upon said activation of said volume delivery means.

12. A method comprising:
accepting a setting for limiting of a reduction in a volume of a drug reservoir that is achieved upon activation of a pressure means upon said reservoir;
defining a rate of a flow of said drug to said reservoir, and
defining said rate of a rate of a flow of said drug from said reservoir when said reservoir is full to be equal to said rate of said flow of said drug to said reservoir.

13. The method as in claim 12, comprising disabling a means for accepting said setting once said setting is accepted.

14. A device comprising
a reservoir suitable to receive a flow of a drug at a pre-designated rate;
a manually operated pump suitable to compress said reservoir an outlet having a pressure-triggered valve suitable to open upon a manual activation of said pump; and
a variable restricting means to limit a volume of said drug expelled from said reservoir upon an activation of said pump, said volume limited to a pre-set volume.
